SEQUENCE LISTING (4 sequences)

SEQ ID NO: 1 - DNA, 2676 bp, Escherichia coli
  source 1..2676: mol_type = other DNA
atggtaggttcgcgctggtataaatttgattttcataaccatactccggcttcgcatgattacaaaattcctgacatcagccccagagagtggcttctggcttatatgaaacagcatgtcgattgtgttgtaatcagcgatcataacagcggagcctgggtcgacgtgttgaagggtgagctggagaatatgtcccgggacgccagcaccggcgacctgccggaatttcggccactgacactctttccgggggttgaactgacagcgaccggtaacgtacatattctggctgtgctgcacacgcacagtacaagtgccgatgtggaaaggcttctggcccagtgcaataataatagccccattccgagtgaagtccctaaccatcagctcgttcttcaactgggccccgccggcatcatcagtaatatccgccgtaatccgaaggctgtttgtattcttgcgcacattgatgcagccaaaggtgtcttaagtctgactaatcaggcagagctcaccgcagcctttcaggaaagtccccatgccgttgagattcgacaccgggtggaggatatcaccgacggaacccgccggcggctgattgataatttaccgtggctacggggctctgatgcgcaccatcctgaacaagccggcatgcgaacctgctggctgaaaatgtcatcccctgattttgacggactcaggcatgcactgctcgatccggaaaactgtgtgctgtttgatcagctccctccggaggaacctgcgtcatatttgcgcagcctgaaattcagaacccgccactgccatcctgtgggtcaggattcggcctcggtggaattcagcccgttctataacgctgtaatcggctcaagaggcagcgggaagtccacgctcattgaaagcattcgtcttgcaatgcgcaaaacagaaggtctcactgcgacccaggggagtaagctggaccagttcattcggacggggatggaagcggattccttcatcgaatgtattttccacaaagaaggcacagatttccggctcagttggcgaccagacagtaagcatgaattacatatcttcagtgacggagaatggatgcctgacagtcactggtcggctgaccgttttccactctcgatttacagccagaaaatgctctatgagctggcttcggatactggtgcattcctgcgcgtctgtgatgagagcccggtggttaacaaacgggcctggaaagagcgctgggatcagctggaaagggaatatctgaatgaacaaatcacgttgcggggcctgcgtgccagacagggaagtgcggattcgctgcggggggaattatcggatgctgaacgtgccgtcagtcagctgcagtcaagcgcctattatccggtttgcagacagctggccctcgccagaaacgagctgtccgcagcaaccttacccctggagcactttgagcggcgtattgcagccattcaggctctggcagaagaaccgctgcagagatccgatatcccgccggaaccttccggtctgctgatggcatttatggcgcgcctgtcatctgtgcaacagcagtatgaccagcggctcaatactctcctggcagaatatgctgcagagctcgcgggtatcaggagagagcaatcttttattgccctccgaacagcagtgagtgaccaggaaacaaatgtagaaagtgaagctgtttccctgcgggccagagggcttaatcccgatgttctcaacgaactgatggcacgctgtgagtcactgaaaaatgagctgagaaattacgacggtcttgatggggcgatctctgcctctgttgcacggtctgagcagttgctggctgaaatgcgtgcccacagaatggcattgacagataaccggaaggcgtttctctcctccctgtcgctcagcgctctggaaatcaaaattcttcccctctgcgccccttatgaagatgttatatctggttaccagacggttaccggcatcagtaattttgccgaacgtatctacgataacagtgacgggagcggattactgagcgactttatcagtgaacgtccgttcagcccgttgcctgccgcaacagagaaaaaatacagggcgctggacgagctgaaagcgctgcatcacagcatccggctggataattcagaggctggggcggggcttcatggttctttccggaatcgtctcaggagtctgaatgaccagcagctggatgccctgcaatgctggtatcctgatgacggcatccacatacgttaccagacccccggggggcagatggaagacattgcctttgcttctccggggcaaaagggagcgagtatgctgcagttcctcttatcctatggcaccgatcctctactactggatcaaccggaggatgacctggactgcctgatgctgagcatgagcgtgatccctgccatcatgtcgaacaagaaacgccggcagctgattatcgtgtcgcactctgcccctatagtggttaacggcgatgcagaatatgttatcagtatgcagcacgatcgcacaggcctgtatccaggactctgcggtgcactgcaggaagctccgatgaaggcactgatatgccgtcaaatggaggggggagaaaaagcgtttcgttcgcgctatgagcgtattcttagctga

SEQ ID NO: 2 - protein, 891 aa, Escherichia coli
MVGSRWYKFDFHNHTPASHDYKIPDISPREWLLAYMKQHVDCVVISDHNSGAWVDVLKGELENMSRDASTGDLPEFRPLTLFPGVELTATGNVHILAVLHTHSTSADVERLLAQCNNNSPIPSEVPNHQLVLQLGPAGIISNIRRNPKAVCILAHIDAAKGVLSLTNQAELTAAFQESPHAVEIRHRVEDITDGTRRRLIDNLPWLRGSDAHHPEQAGMRTCWLKMSSPDFDGLRHALLDPENCVLFDQLPPEEPASYLRSLKFRTRHCHPVGQDSASVEFSPFYNAVIGSRGSGKSTLIESIRLAMRKTEGLTATQGSKLDQFIRTGMEADSFIECIFHKEGTDFRLSWRPDSKHELHIFSDGEWMPDSHWSADRFPLSIYSQKMLYELASDTGAFLRVCDESPVVNKRAWKERWDQLEREYLNEQITLRGLRARQGSADSLRGELSDAERAVSQLQSSAYYPVCRQLALARNELSAATLPLEHFERRIAAIQALAEEPLQRSDIPPEPSGLLMAFMARLSSVQQQYDQRLNTLLAEYAAELAGIRREQSFIALRTAVSDQETNVESEAVSLRARGLNPDVLNELMARCESLKNELRNYDGLDGAISASVARSEQLLAEMRAHRMALTDNRKAFLSSLSLSALEIKILPLCAPYEDVISGYQTVTGISNFAERIYDNSDGSGLLSDFISERPFSPLPAATEKKYRALDELKALHHSIRLDNSEAGAGLHGSFRNRLRSLNDQQLDALQCWYPDDGIHIRYQTPGGQMEDIAFASPGQKGASMLQFLLSYGTDPLLLDQPEDDLDCLMLSMSVIPAIMSNKKRRQLIIVSHSAPIVVNGDAEYVISMQHDRTGLYPGLCGALQEAPMKALICRQMEGGEKAFRSRYERILS

SEQ ID NO: 3 - DNA, 43 bp, synthetic construct
  source 1..43: mol_type = other DNA
aggtcgactctagaggatccttgacaattaatcatcggctcgt

SEQ ID NO: 4 - DNA, 40 bp, synthetic construct
  source 1..40: mol_type = other DNA
taatggtgatggtgatggtggctaagaatacgctcatagc

(19) United States
(12) Patent Application Publication
(10) Pub. No.: US 2025/0074958 A1
(43) Pub. Date: Mar. 6, 2025

(54) APPLICATION OF T1FR PROTEIN IN INHIBITION OF PATHOGENICITY OF ADHERENT-INVASIVE ESCHERICHIA COLI (AIEC)

(71) Applicant: Sichuan University, Chengdu (CN)

(72) Inventors: Hongning WANG, Chengdu (CN); Hongcheng WEI, Chengdu (CN); Changwei LEI, Chengdu (CN); Wenlan YANG, Chengdu (CN); Wei XU, Chengdu (CN)

(21) Appl. No.: 18/949,811

(22) Filed: Nov. 15, 2024

(30) Foreign Application Priority Data
Jan. 23, 2024 (CN) 202410092606.6

(51) Int. Cl. C07K 14/47; C12N 1/20; C12N 15/70; C12R 1/19

(52) U.S. Cl. CPC C07K 14/4703; C12N 1/20; C12N 15/70; C12R 2001/19

(57) ABSTRACT

A T1fr gene coding a type 1 fimbrial repressor (T1FR) protein, whose nucleotide sequence is shown in SEQ ID NO: 1. An amino acid sequence of the T1FR protein is shown in SEQ ID NO: 2. Applications of the T1FR protein in the inhibition of fimbrial growth, and intestinal colonization and pathogenicity of an adherent-invasive Escherichia coli (AIEC) strain are further provided.

### REFERENCE TO AN ELECTRONIC SEQUENCE LISTING

The contents of the electronic sequence listing (Name: SequenceListing.xml; Size: 8,078 bytes; and Date of Creation: Nov. 6, 2024) is herein incorporated by reference in its entirety.

### CROSS-REFERENCE TO RELATED APPLICATIONS

This application claims the benefit of priority from Chinese Patent Application No. 202410092606.6, filed on Jan. 23, 2024. The content of the aforementioned application, including any intervening amendments made thereto, is incorporated herein by reference in its entirety.

### TECHNICAL FIELD

This application relates to application of T1FR protein, and more particularly to an application of the T1FR protein in the inhibition of pathogenicity of adherent-invasive Escherichia coli (AIEC).

### BACKGROUND

Adherent-invasive Escherichia coli (AIEC), as a pathotype of E. coli, can specifically adhere to intestinal epithelial cells to colonize the intestinal mucosa, and also survive and replicate within macrophages. AIEC penetrates the mucus layer by binding its type 1 fimbriae to the carcinoembryonic antigen-related cell adhesion molecule 6 (CEACAM6) of the intestinal epithelial cell (IEC), and the expression of CEACAM6 is significantly upregulated in the intestinal inflammatory regions of patients with Crohn's disease (CD). The AIEC adhering to the IEC can promote the release of inflammatory factors by activating the NF-κB pathway, thereby inducing the intestinal inflammation. Additionally, it has been extensively demonstrated that AIEC is involved in triggering intestinal inflammation in CD patients. After adhering to the IEC, the polysaccharide matrix secreted by the will cover the bacterial cells to form a biofilm that exhibits strong resistance to antibiotics and host immunity, thereby creating a stable internal environment for the persistent colonization of AIEC. Moreover, the current clinical broad-spectrum antibiotic drugs will damage the intestinal flora and facilitate the AIEC adhesion and colonization, exacerbating the occurrence of intestinal inflammation while promoting the evolution of bacterial drug-resistance.

Considering that the fimbriae play an important role in the adhesion and colonization of AIEC to the intestinal epithelial cells, inhibiting the fimbriae-mediated adhesion process has been considered as an effective strategy for preventing and treating AIEC-induced intestinal inflammation.

### SUMMARY

An object of the present disclosure is to provide a T1FR protein capable of inhibiting type 1 fimbriae of Escherichia coli to address the issues in the prior art. The T1FR protein provided herein can effectively suppress the pathogenicity of AIEC to the mouse intestinal tract, offering a reference scheme for the clinical prevention and treatment of Crohn's disease.

Technical solutions of the present disclosure are described as follows.

In a first aspect, this application provides a T1fr
gene coding a type 1 fimbrial repressor (T1FR) protein,
whose nucleotide sequence is represented by SEQ ID NO: 1:
ATGGTAGGTTCGCGCTGGTATAAATTTGATTTTCATAACCATACTCCGGC
TTCGCATGATTACAAAATTCCTGACATCAGCCCCAGAGAGTGGCTTCTGGC
TTATATGAAACAGCATGTCGATTGTGTTGTAATCAGCGATCATAACAGCGGA
GCCTGGGTCGACGTGTTGAAGGGTGAGCTGGAGAATATGTCCCGGGACGC
CAGCACCGGCGACCTGCCGGAATTTCGGCCACTGACACTCTTTCCGGGGGT
TGAACTGACAGCGACCGGTAACGTACATATTCTGGCTGTGCTGCACACGCA
CAGTACAAGTGCCGATGTGGAAAGGCTTCTGGCCCAGTGCAATAATAATAG
CCCCATTCCGAGTGAAGTCCCTAACCATCAGCTCGTTCTTCAACTGGGCCC
CGCCGGCATCATCAGTAATATCCGCCGTAATCCGAAGGCTGTTTGTATTCTT
GCGCACATTGATGCAGCCAAAGGTGTCTTAAGTCTGACTAATCAGGCAGAG
CTCACCGCAGCCTTTCAGGAAAGTCCCCATGCCGTTGAGATTCGACACCGG
GTGGAGGATATCACCGACGGAACCCGCCGGCGGCTGATTGATAATTTACCG
TGGCTACGGGGCTCTGATGCGCACCATCCTGAACAAGCCGGCATGCGAACC
TGCTGGCTGAAAATGTCATCCCCTGATTTTGACGGACTCAGGCATGCACTG
CTCGATCCGGAAAACTGTGTGCTGTTTGATCAGCTCCCTCCGGAGGAACCT
GCGTCATATTTGCGCAGCCTGAAATTCAGAACCCGCCACTGCCATCCTGTG
GGTCAGGATTCGGCCTCGGTGGAATTCAGCCCGTTCTATAACGCTGTAATCG
GCTCAAGAGGCAGCGGGAAGTCCACGCTCATTGAAAGCATTCGTCTTGCA
ATGCGCAAAACAGAAGGTCTCACTGCGACCCAGGGGAGTAAGCTGGACCA
GTTCATTCGGACGGGGATGGAAGCGGATTCCTTCATCGAATGTATTTTCCAC
AAAGAAGGCACAGATTTCCGGCTCAGTTGGCGACCAGACAGTAAGCATGA
ATTACATATCTTCAGTGACGGAGAATGGATGCCTGACAGTCACTGGTCGGC
TGACCGTTTTCCACTCTCGATTTACAGCCAGAAAATGCTCTATGAGCTGGCT
TCGGATACTGGTGCATTCCTGCGCGTCTGTGATGAGAGCCCGGTGGTTAAC
AAACGGGCCTGGAAAGAGCGCTGGGATCAGCTGGAAAGGGAATATCTGAA
TGAACAAATCACGTTGCGGGGCCTGCGTGCCAGACAGGGAAGTGCGGATT
CGCTGCGGGGGGAATTATCGGATGCTGAACGTGCCGTCAGTCAGCTGCAGT
CAAGCGCCTATTATCCGGTTTGCAGACAGCTGGCCCTCGCCAGAAACGAGC
TGTCCGCAGCAACCTTACCCCTGGAGCACTTTGAGCGGCGTATTGCAGCCA
TTCAGGCTCTGGCAGAAGAACCGCTGCAGAGATCCGATATCCCGCCGGAA
CCTTCCGGTCTGCTGATGGCATTTATGGCGCGCCTGTCATCTGTGCAACAGC
AGTATGACCAGCGGCTCAATACTCTCCTGGCAGAATATGCTGCAGAGCTCG
CGGGTATCAGGAGAGAGCAATCTTTTATTGCCCTCCGAACAGCAGTGAGTG
ACCAGGAAACAAATGTAGAAAGTGAAGCTGTTTCCCTGCGGGCCAGAGGG
CTTAATCCCGATGTTCTCAACGAACTGATGGCACGCTGTGAGTCACTGAAA
AATGAGCTGAGAAATTACGACGGTCTTGATGGGGCGATCTCTGCCTCTGTT
GCACGGTCTGAGCAGTTGCTGGCTGAAATGCGTGCCCACAGAATGGCATTG
ACAGATAACCGGAAGGCGTTTCTCTCCTCCCTGTCGCTCAGCGCTCTGGAA
ATCAAAATTCTTCCCCTCTGCGCCCCTTATGAAGATGTTATATCTGGTTACCA
GACGGTTACCGGCATCAGTAATTTTGCCGAACGTATCTACGATAACAGTGAC
GGGAGCGGATTACTGAGCGACTTTATCAGTGAACGTCCGTTCAGCCCGTTG
CCTGCCGCAACAGAGAAAAAATACAGGGCGCTGGACGAGCTGAAAGCGC
TGCATCACAGCATCCGGCTGGATAATTCAGAGGCTGGGGCGGGGCTTCATG
GTTCTTTCCGGAATCGTCTCAGGAGTCTGAATGACCAGCAGCTGGATGCCC
TGCAATGCTGGTATCCTGATGACGGCATCCACATACGTTACCAGACCCCCGG
GGGGCAGATGGAAGACATTGCCTTTGCTTCTCCGGGGCAAAAGGGAGCGA
GTATGCTGCAGTTCCTCTTATCCTATGGCACCGATCCTCTACTACTGGATCAA
CCGGAGGATGACCTGGACTGCCTGATGCTGAGCATGAGCGTGATCCCTGCC
ATCATGTCGAACAAGAAACGCCGGCAGCTGATTATCGTGTCGCACTCTGCC
CCTATAGTGGTTAACGGCGATGCAGAATATGTTATCAGTATGCAGCACGATC
GCACAGGCCTGTATCCAGGACTCTGCGGTGCACTGCAGGAAGCTCCGATG
AAGGCACTGATATGCCGTCAAATGGAGGGGGGAGAAAAAGCGTTTCGTTC
GCGCTATGAGCGTATTCTTAGCTGA.
In a second aspect, this application provides a T1FR
protein expressed by the T1fr gene, whose amino
acid sequence is represented by SEQ ID NO: 2:
MVGSRWYKFDFHNHTPASHDYKIPDISPREWLLAYMKQHVDCVVISDH
NSGAWVDVLKGELENMSRDASTGDLPEFRPLTLFPGVELTATGNVHILAVLHT
HSTSADVERLLAQCNNNSPIPSEVPNHQLVLQLGPAGIISNIRRNPKAVCILAHI
DAAKGVLSLTNQAELTAAFQESPHAVEIRHRVEDITDGTRRRLIDNLPWLRGS
DAHHPEQAGMRTCWLKMSSPDFDGLRHALLDPENCVLFDQLPPEEPASYLRS
LKFRTRHCHPVGQDSASVEFSPFYNAVIGSRGSGKSTLIESIRLAMRKTEGLTAT
QGSKLDQFIRTGMEADSFIECIFHKEGTDFRLSWRPDSKHELHIFSDGEWMPD
SHWSADRFPLSIYSQKMLYELASDTGAFLRVCDESPVVNKRAWKERWDQLER
EYLNEQITLRGLRARQGSADSLRGELSDAERAVSQLQSSAYYPVCRQLALARN
ELSAATLPLEHFERRIAAIQALAEEPLQRSDIPPEPSGLLMAFMARLSSVQQQY
DQRLNTLLAEYAAELAGIRREQSFIALRTAVSDQETNVESEAVSLRARGLNPDV
LNELMARCESLKNELRNYDGLDGAISASVARSEQLLAEMRAHRMALTDNRK
AFLSSLSLSALEIKILPLCAPYEDVISGYQTVTGISNFAERIYDNSDGSGLLSDFI
SERPFSPLPAATEKKYRALDELKALHHSIRLDNSEAGAGLHGSFRNRLRSLND
QQLDALQCWYPDDGIHIRYQTPGGQMEDIAFASPGQKGASMLQFLLSYGTDP
LLLDQPEDDLDCLMLSMSVIPAIMSNKKRRQLIIVSHSAPIVVNGDAEYVISMQ
HDRTGLYPGLCGALQEAPMKALICRQMEGGEKAFRSRYERILS.

In a third aspect, this application provides a method for preparing the T1FR protein, comprising:
(1) amplifying the T1fr gene with a pEC51 plasmid (Accession NO. OQ230786.1) as a template (Forward primer-AGGTCGACTCTAGAGGATCCTTGACAATTAATCATCGGCTCGT (SEQ ID NO: 3), Reverse primer-TAATGGTGATGGTGATGGTGGCTAAGAATACGCTCATAGC (SEQ ID NO: 4)), followed by purification; and
(2) inserting the T1fr gene into an expression vector plasmid to construct a recombinant plasmid, and transforming the recombinant plasmid into a host bacterial cell for expression.

In some embodiments, in step (2), the expression vector plasmid is pUC19, and the host bacterial cell is an adherent-invasive Escherichia coli (AIEC).

In a fourth aspect, this application provides a method for inhibiting the growth of fimbriae of an adherent-invasive Escherichia coli strain, comprising:
transforming the T1fr gene into the adherent-invasive Escherichia coli strain.

In a fifth aspect, this application provides a method for inhibiting the intestinal colonization of an adherent-invasive Escherichia coli strain, comprising:
transforming the T1fr gene into the adherent-invasive Escherichia coli strain.

In a sixth aspect, this application provides a method for inhibiting the intestinal pathogenicity of an adherent-invasive Escherichia coli strain, comprising:
transforming the T1fr gene into the adherent-invasive Escherichia coli strain.

Compared to the prior art, the present disclosure has the following beneficial effects.
1. The present disclosure provides a T1FR protein encoded by the nucleotide sequence of SEQ ID NO: 1, and a preparation method thereof. Studies have demonstrated that this T1FR protein can inhibit the growth of type 1 fimbriae of Escherichia coli, and effectively suppress the intestinal colonization and pathogenicity of adherent-invasive Escherichia coli (AIEC) in the mouse. These results suggest that blocking the adhesion process of bacterial fimbriae to tissues and organs through biological factors can significantly reduce the bacterial pathogenicity, thereby offering a valuable reference strategy for the effective management of bacterial infectious diseases.
2. The present disclosure also provides applications of the T1FR protein in the inhibition of fimbrial growth, and intestinal colonization and pathogenicity of the adherent-invasive Escherichia coli strain.

### BRIEF DESCRIPTION OF THE DRAWINGS

FIG. 1 schematically shows the inhibition of a T1FR protein against the pathogenicity of an adherent-invasive Escherichia coli (AIEC) strain;

FIG. 2 schematically shows a structure of the T1FR protein;

FIGS. 3A-3B schematically show construction of a recombinant plasmid;

FIGS. 4A-B are fluorescence microscopic images of a LF82 strain transformed with a plasmid and a LF82 strain transformed with a recombinant plasmid pUC19+T1fr;

FIG. 4C schematically shows a verification result of plasmid transformation in the LF82 strain by PCR;

FIG. 4D schematically shows the verification result of T1FR protein expression in the LF82 strain by Western blot;

FIGS. 5A-5B are transmission electron microscope images of fimbriae from a LF82/pUC19 strain;

FIGS. 5C-5D are transmission electron microscope images of fimbriae from a LF82/pUC19+T1fr strain;

FIGS. 6A-6C schematically show adhesion of the LF82/pUC19 strain to eukaryotic cells using an upright fluorescence microscope;

FIGS. 6D-6F schematically show adhesion of the LF82/pUC19+T1fr strain to eukaryotic cells using the upright fluorescence microscope;

FIG. 6G schematically shows fluorescence intensity observed through the upright fluorescence microscope;

FIGS. 7A-7D schematically show a biofilm image of the LF82/pUC19 strain observed using a fluorescence microscope (Leica DM4 B);

FIGS. 7E-7H schematically show the biofilm image of the LF82/pUC19+T1fr strain observed using the fluorescence microscope (Leica DM4 B);

FIG. 8A schematically shows a survival rate of mice infected with AIEC;

FIGS. 8B-8G schematically show a result of colony count and dilution factor;

FIG. 8H schematically shows a quantitative analysis of the colonization level of AIEC in the mouse intestine;

FIGS. 9A-9H schematically show an intestinal pathological section in mice infected with AIEC;

FIG. 9I schematically shows a result of intestinal cytokine detection in mice infected with AIEC; and

FIG. 9J schematically shows diversity analysis of 16S rRNA in the intestinal microflora of mice infected with AIEC.

### DETAILED DESCRIPTION OF EMBODIMENTS

The present disclosure will be described in detail below in conjunction with the accompanying figures.

To clarify the objects, technical solutions, and advantages of the present disclosure, the following detailed description is provided in conjunction with the accompanying figures and embodiments. It should be noted that the specific embodiments described herein are merely illustrative, and are not intended to limit the present disclosure.

### EXAMPLE 1

### 1. Construction and Transformation of T1FR Protein Expression Vector

A nucleotide sequence of a T1fr gene coding a type 1 fimbrial repressor (T1FR) protein was shown in SEQ ID NO: 1.

The T1FR protein encoded by the T1fr gene had an amino acid sequence represented by SEQ ID NO: 2.

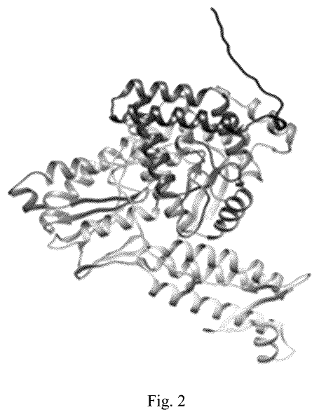

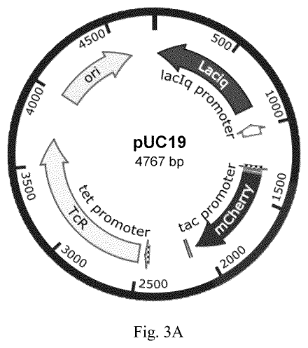

A structure of the T1FR protein was predicted using AlphaFold, as shown in FIG. 2.

A pUC19 plasmid containing the T1fr gene (pUC19+T1fr) was constructed by homologous recombination through the following steps.

### (1) Amplification of the T1fr Gene

The T1fr gene was amplified with a pEC51 plasmid (Accession NO. OQ230786.1) as a template (Forward primer-AGGTCGACTCTAGAGGATCCTTGACAATTAATCATCGGCTCGT (SEQ ID NO: 3); Reverse primer-TAATGGTGATGGTGATGGTGGCTAAGAATACGCTCATAGC (SEQ ID NO: 4)), and then the amplified product was collected and purified.
(2) Recombinant Plasmid Construction

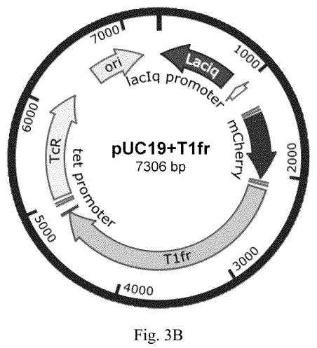

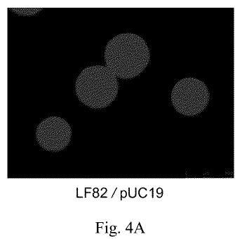

The nucleotide sequence as shown in SEQ ID NO: 1 (i.e., the T1fr gene) was cloned into a pUC19 plasmid to construct a recombinant plasmid pUC19+T1fr, as illustrated in FIGS. 3A-3B.

The pUC19+T1fr plasmid obtained in step (2) was introduced into a LF82 strain to obtain the LF82 strain with overexpression of the T1FR protein.

### 2. Analysis of T1FR Protein Expression in LF82 Strain

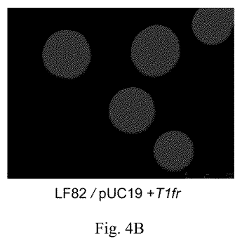

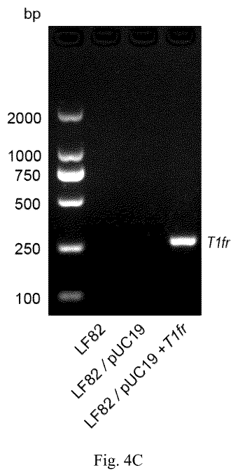

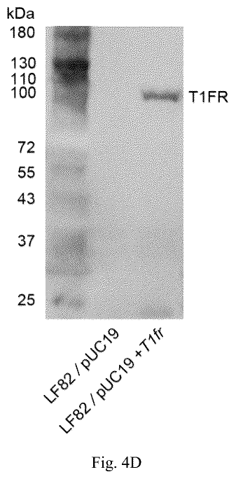

As shown in FIGS. 4A-4D, the wild-type LF82 strain was cultured to the logarithmic phase at 37° C. under shaking, and then respectively transformed with the pUC19 plasmid and the pUC19+T1fr plasmid by electrotransformation, designated as LF82/pUC19 strain and LF82/pUC19+T1fr strain. The expression of the fluorescent protein in the plasmid was observed using an upright fluorescence microscope (Leica DM4 B), as illustrated in FIGS. 4A-4B, confirming the successful transformation of the plasmid into the LF82 strain. FIG. 4C showed the verification result of plasmid transformation in the LF82 strain by PCR, and FIG. 4D demonstrated the expression of the T1FR protein in the LF82 strain by Western blot.

The expression of T1FR protein was demonstrated through various analysis methods, indicating the successful expression of T1FR protein in the recombinant LF82 strain.

### 3. Analysis of Inhibition Effect of T1FR Protein on Fimbriac of LF82 Strain

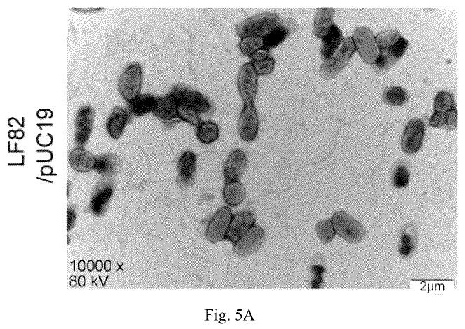

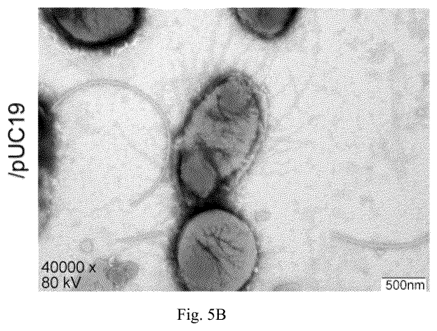

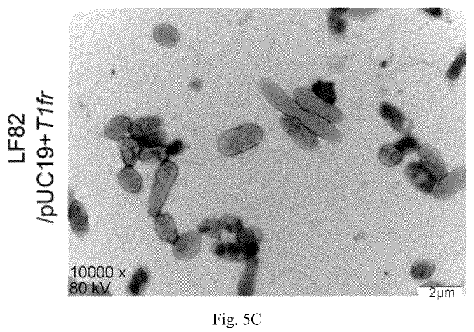

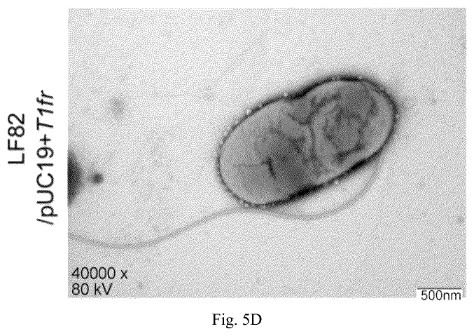

LF82/pUC19 strain and LF82/pUC19+T1fr strain were inoculated respectively onto Mueller-Hinton Agar (MHA) plates and cultured overnight at 37° C. Single colonies were picked, stained with 2% phosphotungstic acid for 3-10 s, naturally dried at room temperature, and observed using a transmission electron microscope (JEM-1400FLASH) for the fimbriac, as illustrated in FIGS. 5A-5D.

As observed through the transmission electron microscope, the T1FR protein could effectively inhibit the fimbrial growth in the LF82 strain.

### 4. Inhibition of T1FR Protein Against the Adhesion of LF82 Strain to Eukaryotic Cells

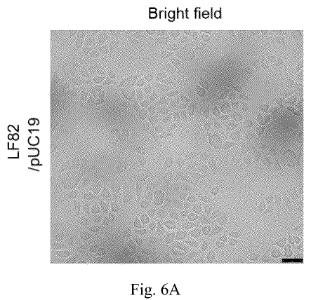

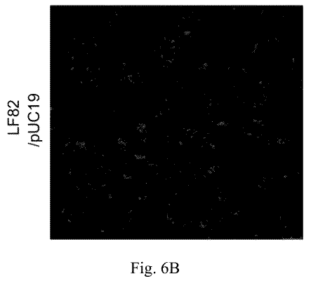

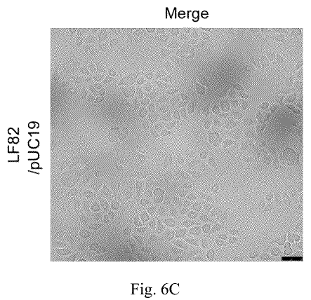

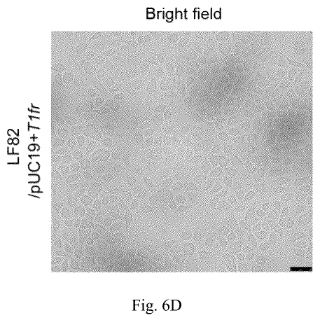

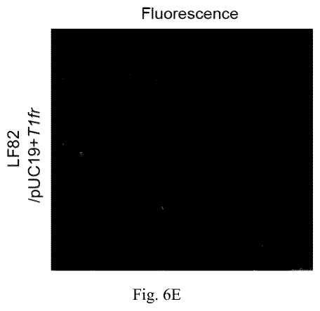

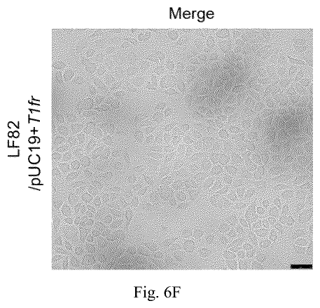

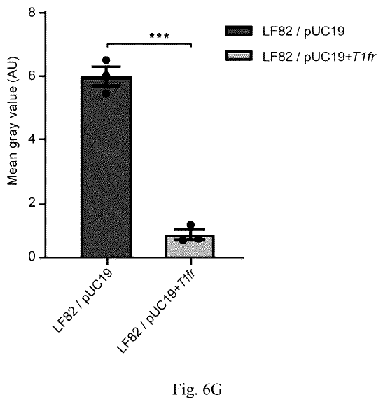

As shown in FIGS. 6A-6F, HIEC-6 epithelial cells were cultured in Roswell Park Memorial Institute (RPMI) 1640 medium (Sigma-Aldrich) at 37° C. with 5% CO₂. The cultured cells were dispensed into a 24-well plate, with approximately 4×10⁵ cells per well, and continued to be cultured for 16 h. Non-adherent cells were removed using phosphate-buffered saline (PBS). The LF82/pUC19 strain and LF82/pUC19+T1fr strain were cultured overnight, centrifuged at 10,000 rpm to collect the bacterial cells, which were then resuspended in the RPMI 1640 medium. The bacterial suspension was added to the 24-well plate at a multiplicity of infection (MOI) ratio of 10, and incubated at 37° C. for 3 h. The liquid in the wells was removed, and the unattached cells were washed with PBS. As shown in FIGS. 6A-6F, the adhesion of the LF82/pUC19 strain and LF82/pUC19+T1fr strain to eukaryotic cells was evaluated using the upright fluorescence microscope, and the microscopic observation results demonstrated that the type 1 fimbriae of LF82/pUC19+T1fr strain was suppressed.

The fluorescence intensity observed through the upright fluorescence microscope was analyzed using ImageJ software (FIG. 6G), and the results indicated that T1FR protein can inhibit the adhesion of the LF82 strain to eukaryotic cells.

### 5. Inhibition of LF82 Strain Biofilm Formation by T1FR Protein

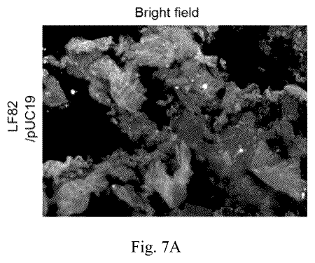

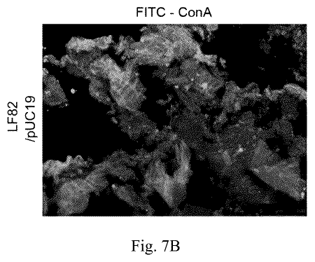

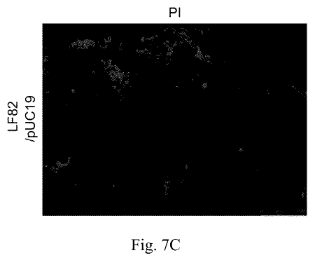

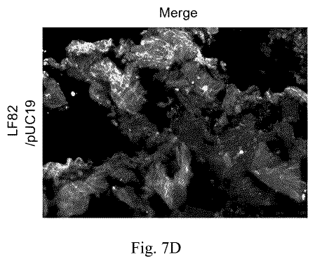

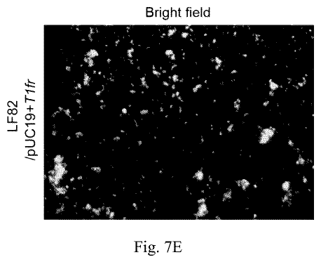

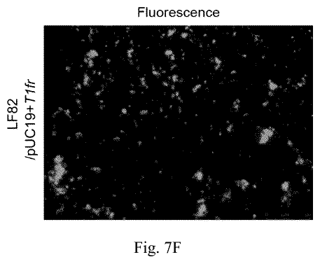

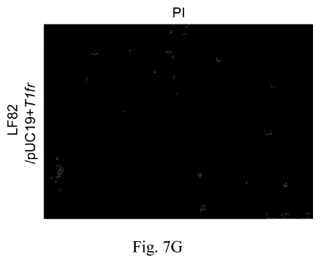

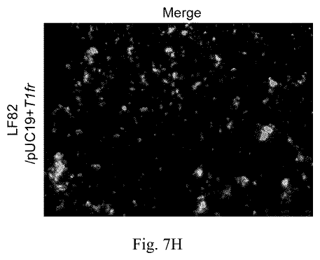

As shown in FIGS. 7A-7H, the LF82/pUC19 strain and the LF82/pUC19+T1fr strain were respectively cultured overnight at 37° C. under shaking, and then diluted to 10⁸ CFU/mL. A slide was placed into the diluted bacterial solution, and incubated for 24 h. The biofilm attached to the slide was washed with PBS, fixed with 2.5% glutaraldehyde, washed again with PBS, stained with Fluorescein Isothiocyanate-Concanavalin A (FITC-ConA) at 4° C. in the dark for 30 min. The slide was washed again with PBS and stained with propidium iodide (PI) away from light for 15 min. The slide was washed with PBS to remove excess dye, and dried. The biofilms of both strains were observed and analyzed using the upright fluorescence microscope (Leica DM4 B). The results revealed that the T1FR protein could effectively weaken the biofilm formation ability of the LF82 strain.

6. Survival Rate of Mice Infected with Adherent-Invasive Escherichia coli (AIEC) and Intestinal Colonization Testing of AIEC in the Mice

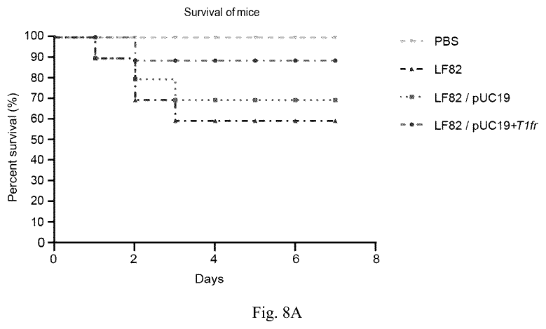

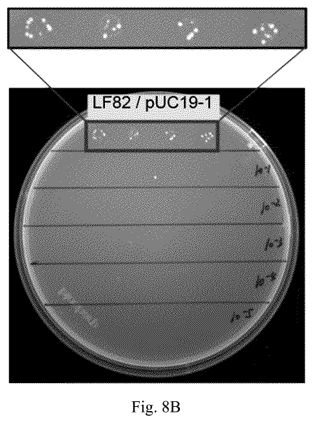

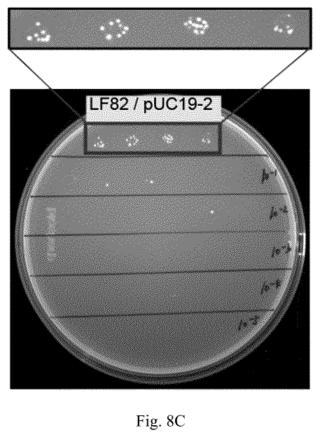

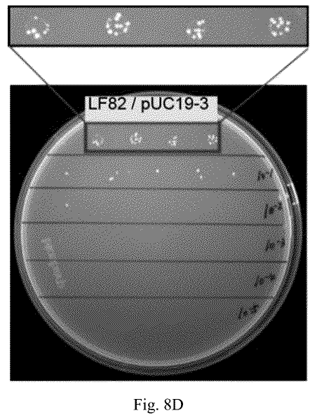

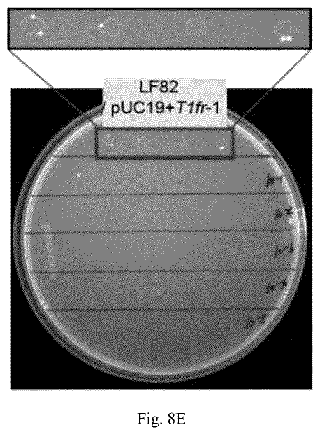

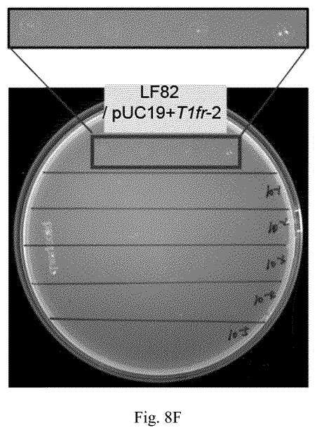

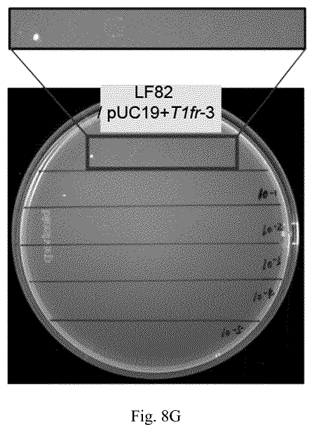

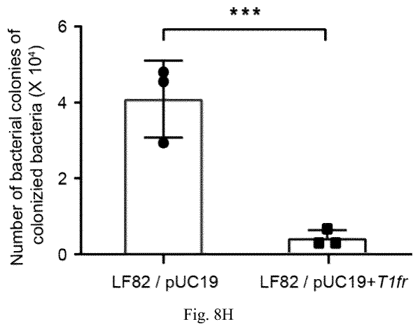

Five-week-old mice were randomly divided into four groups (PBS, LF82, LF82/pUC19, and LF82/pUC19+T1fr), with ten mice in each group. The mice from the LF82, LF82/pUC19, and LF82/pUC19+T1fr groups were intragastrically administered with AIEC (109 CFU/day) for three consecutive days, while the PBS group received the same volume of PBS. The mice were then raised for seven days to evaluate their survival rates. The results indicated that the T1FR protein could enhance the survival rate of mice infected with the LF82 strain as shown in FIG. 8A.

The complete colon and rectum of the mice were excised to remove the contents, and added with a 4° C. PBS (0.1 g/mL) and homogenized using a benchtop tissue homogenizer to prepare a tissue homogenate, which further underwent 10-fold serial dilution. 1 μL of the diluted homogenate was inoculated onto a LB agar plate containing tetracycline (5 μg/mL) to assess the colonization of AIEC in the mouse intestine, based on the number of colonies on the plates and the dilution factor, as shown in FIGS. 8B-8G. The colonization level of AIEC in the mouse intestine was quantitatively analyzed, and the results were presented in FIG. 8H, indicating that the T1FR protein significantly reduced the colonization of the LF82 strain in the mouse intestine.

7. Intestinal Pathological Analysis of the Mice Infected with AIEC

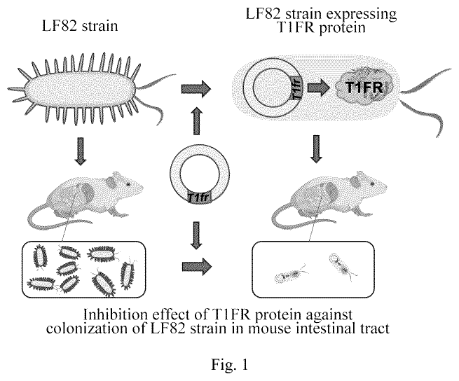

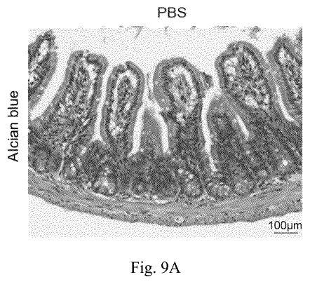

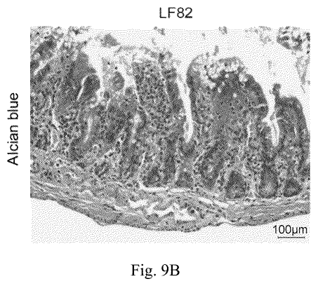

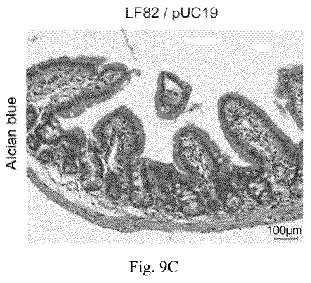

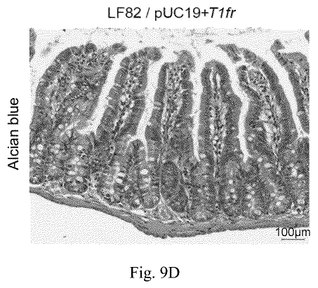

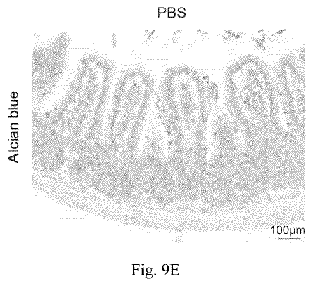

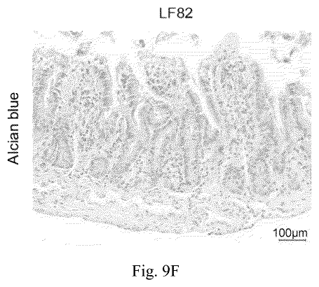

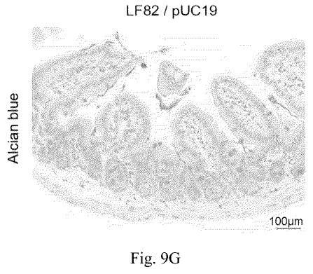

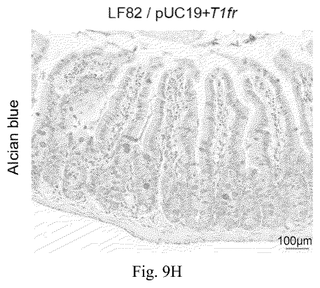

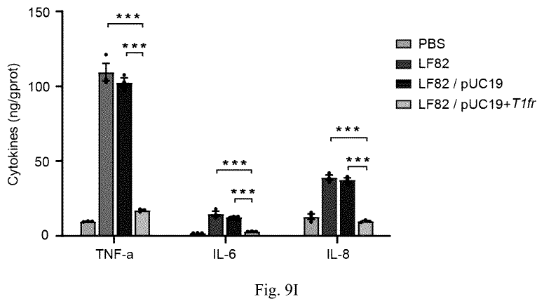

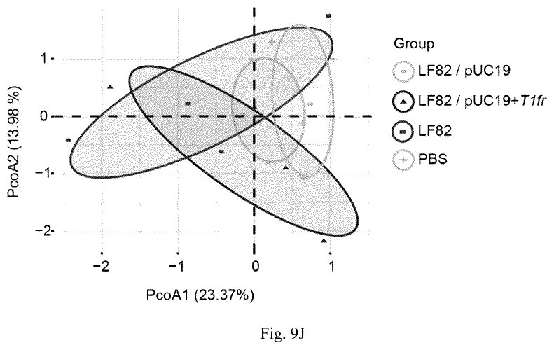

As shown in FIG. 1, the T1FR could inhibit the pathogenicity of an adherent-invasive Escherichia coli (AIEC) strain. FIGS. 9A-9H showed an intestinal pathological section of mice infected with AIEC. The colon of the mice was collected, fixed in a fixative solution for 24 h, dehydrated, embedded with paraffin and cut to prepare 5 μm tissue sections. Then the 5 μm tissue sections were stained with hematoxylin-eosin (H&E) and Alcian blue, and scanned using a panoramic tissue section scanning system (Olympus VS200). The results indicated that the T1FR protein could alleviate the LF82 strain-induced intestinal pathological damage.

A result of intestinal cytokine detection in mice infected with AIEC was presented in FIG. 91. The total protein content in the intestine was measured as instructed by the total protein assay kit (Nanjing Jiancheng Bioengineering Institute). The levels of the cytokines TNFα, IL-6, and IL-8 in the intestine were determined as instructed by the enzyme-linked immunosorbent assay (ELISA) kit (Jiangsu Meimian Industrial Co., Ltd). The measured values were normalized to total protein content and subjected to statistical analysis. The results indicated that the T1FR protein could inhibit the production of inflammatory factors in intestinal tissues induced by the LF82 strain. A diversity analysis of 16S rRNA in the intestinal microbiota of mice infected with AIEC, as shown in FIG. 9J. Intestinal contents were collected from the mice, and the DNA was extracted as instructed by the E.Z.N.A.™ Mag-Bind Soil DNA Kit (OMEGA), and sequenced by Sangon Biotech (Shanghai) Co., Ltd. Non-redundant sequences with an identity greater than 97% were clustered into operational taxonomic units (OTUs) using UPARSE. Based on the OTU clustering results, the diversity analysis was conducted, and the results indicated that the T1FR protein had less impact on the intestinal microecology.

The above experimental results demonstrated that the T1FR protein can effectively inhibit the pathogenicity of AIEC strains in the mouse intestine.

The embodiments described above are merely illustrative of the present disclosure, and are not intended to limit the scope of the present disclosure. It should be understood that various changes or substitutions made by those of ordinary skill in the art without departing from the spirit of the present disclosure shall fall within the scope of the present disclosure defined by the appended claims.

What is claimed is:

1. A T1fr gene coding a type 1 fimbrial repressor (T1FR) protein, wherein a nucleotide sequence of the T1fr gene consists of SEQ ID NO: 1.

2. A T1FR protein expressed by the T1fr gene of claim 1, wherein an amino acid sequence of the T1FR protein consists of SEQ ID NO: 2.

3. A method for preparing the T1FR protein of claim 2, comprising:
(1) amplifying the T1fr gene with a pEC51 plasmid as a template, followed by purification; and
(2) inserting the T1fr gene into an expression vector plasmid to construct a recombinant plasmid, and transforming the recombinant plasmid into a host bacterial cell for expression.

4. The method of claim 3, wherein in step (2), the expression vector plasmid is pUC19, and the host bacterial cell is an adherent-invasive Escherichia coli (AIEC) cell.

5. A method for inhibiting fimbrial growth of an adherent-invasive Escherichia coli strain, comprising:
transforming the T1fr gene of claim 1 into the adherent-invasive Escherichia coli strain.

6. A method for inhibiting intestinal colonization and intestinal pathogenicity of an adherent-invasive Escherichia coli strain, comprising:
transforming the T1fr gene of claim 1 into the adherent-invasive Escherichia coli strain.
